(19) United States
(12) Patent Application Publication
(10) Pub. No.: US 2025/0387925 A1
(43) Pub. Date: Dec. 25, 2025

(54) ROBOT DEVICE

(71) Applicant: Karl Storz SE & Co. KG, Tuttlingen (DE)

(72) Inventors: Martin Hoffmann, Berlin (DE); Matthias Borutta, Berlin (DE)

(73) Assignee: Karl Storz SE & Co. KG, Tuttlingen (DE)

(21) Appl. No.: 19/241,131

(22) Filed: Jun. 17, 2025

(30) Foreign Application Priority Data
Jun. 21, 2024 (DE) 102024117645.8

(51) Int. Cl. B25J 15/04; A61B 34/35; B25J 9/10; B25J 13/06; B25J 18/00

(52) U.S. Cl. CPC B25J 15/0408; A61B 34/35; B25J 9/1045; B25J 13/06; B25J 18/00

(57) ABSTRACT

A robotic apparatus comprises a proximal movement arm which is pivotable about at least one pivot axis, a distal movement arm which is articulately coupled to the proximal movement arm, wherein the distal movement arm is rotatable about at least one rotation axis relative to the proximal movement arm, wherein the rotation axis extends at an angle to the pivot axis.

### CROSS-REFERENCE TO RELATED APPLICATIONS

This application claims priority of German Patent Application No. DE 10 2024 117 645.8 filed on Jun. 21, 2024, the contents of which are incorporated herein.

### TECHNICAL FIELD

The present invention relates to a robotic apparatus, in particular as part of a medical robotic system.

### BACKGROUND

Telerobotic systems are playing an increasingly important role in medicine, enabling surgeons to perform minimally invasive operations with precision. These systems essentially comprise two main components: the master unit and the slave unit. The master unit is usually a control device operated by a surgeon, and detects movements and inputs from the surgeon. The slave unit is usually at least one robot-assisted effector that precisely mimics the movements of the master unit and performs the surgical procedure. The surgeon controls the slave unit via the master unit, with the system providing haptic feedback to give the surgeon a feel for, for example, tissue resistance and structures during a surgical procedure.

Conventional master and/or slave units of robotic systems have a high inertia due to their design and/or are limited in their working space, i.e. the three-dimensional space within which the master and/or slave units can be moved, which significantly impairs the precision and/or user-friendliness of such a robotic system.

### SUMMARY

The invention is based on in particular—but not limited to—the object of advantageously further developing a robotic apparatus for use in a medical robotic system, in particular with regard to reducing the inertia of the robotic apparatus while at the same time maintaining a high degree of freedom of movement. Furthermore, it is a particular object of the present invention to provide a precise and user-friendly robotic apparatus with optimized properties so that in particular the ratio of the size of the robotic apparatus to the working space can be improved.

This object is achieved according to the invention by the features of the independent claims. Developments of the invention can be found in the dependent claims.

The invention relates to a robotic apparatus comprising:
a proximal movement arm which is pivotable about at least one pivot axis;
a distal movement arm which is articulately coupled to the proximal movement arm, wherein the distal movement arm is rotatable about at least one rotation axis relative to the proximal movement arm, wherein the rotation axis extends at an angle to the pivot axis.

Such a design makes it possible to provide an advantageously further developed robotic apparatus. In particular, such a robotic apparatus has low inertia while maintaining a high degree of freedom of movement. In addition, a robotic apparatus can be provided which has an advantageous ratio of the size of the robotic apparatus to its working space, wherein in particular the precision and/or the user-friendliness of the robotic apparatus can be increased.

The term “robotic apparatus” is to be understood as meaning, in particular, a component of a robotic system, in particular a subassembly and/or a structural and/or functional component of a robotic system. The robotic apparatus may, for example, be configured to reliably capture and/or execute precise movements during a surgical procedure. A robotic apparatus can, for example, be part of a master and/or slave unit of a teleoperation system.

The term “configured” shall be understood to mean specifically programmed, provided, adapted, designed and/or equipped. The fact that an object is provided for a specific function shall further be understood, in particular, to mean that the object fulfills and/or executes this specific function in at least one application and/or operating state.

A movement arm is to be understood herein in particular to mean a preferably elongated and/or rigid mechanical structure, which is in particular part of a robot arm or an input arm and can be configured to be moved precisely and/or intuitively. A movement arm can have a limited number of degrees of freedom. The structure of the movement arm can vary depending on requirements and applications. The structure of the movement arm can be one-piece, monolithic and/or multi-piece. The shape of the movement arm can be designed in such a way that optimal freedom of movement is possible. In order to ensure adequate mechanical properties, in particular with regard to load-bearing capacity and/or stability of the movement arm, the latter can preferably be made of a solid and/or rigid material such as metal, high-performance polymers and/or composite materials or another advantageous combination of materials. The mechanical structure of the movement arm may have material recesses that can contribute to weight reduction and/or improvement of its mechanical properties.

The proximal movement arm may be a structure located closer to a base of the robotic apparatus and/or extending from the base. The base can be understood in the context of this invention as a basic construction on and/or to which the proximal movement arm can be and/or is mounted directly and/or indirectly and/or from which the proximal movement arm extends. Spatially speaking, the proximal movement arm can represent a lower and/or rear movement arm of the robotic apparatus. A proximal and/or lower movement arm may in particular refer to a movement arm that is arranged closer to the base of the robotic apparatus than is the distal movement arm.

The distal movement arm can be understood as a movement arm of the robotic apparatus that is located further away from the base of the robotic apparatus compared to the proximal movement arm.

The statement that the proximal movement arm is pivotable about at least one pivot axis is intended to mean that the proximal movement arm can be moved in one or more directions in order to reach different positions. The at least one pivot axis can run in particular in the horizontal direction. The pivot axis can primarily be a reference axis that refers to a single degree of freedom that allows a pivoting movement of a component about that pivot axis. The pivoting movement of the proximal movement arm can be carried out in predefined angular steps, but is preferably almost stepless and particularly preferably stepless. The connection point that allows the proximal movement arm to pivot around the pivot axis can also be referred to as the articulated joint. The proximal movement arm can be pivotable about the pivot axis by at least 45°, preferably by at least 90° and particularly preferably by at least 120°.

The statement that the distal movement arm is articulately coupled to the proximal movement arm should be understood within the context of this invention to mean that the proximal and distal movement arms are coupled to one another in such a way that the proximal and distal movement arms are movable independently of one another and/or relative to one another, while the proximal movement arm remains directly or indirectly connected to the distal movement arm.

The statement that the distal movement arm is rotatable about at least one rotation axis relative to the proximal movement arm is intended to mean that the distal movement arm can be moved in one or more directions to reach different positions. The distal movement arm may be rotatable about a reference axis that is different from the at least one pivot axis. The rotation axis can in particular run perpendicular to the distal movement arm. In particular, it would be conceivable for the rotation axis to maintain its angular orientation relative to a direction of gravity for any pivot position of the proximal movement arm, and preferably to remain aligned parallel to the direction of gravity. The rotation axis can primarily be a reference axis that relates to a single degree of freedom that makes rotary and/or rotational movement of a component about that rotation axis possible. The rotational movement of the distal movement arm can take place in predefined angular steps, but is preferably almost stepless and particularly preferably stepless. The connection point that allows the distal movement arm to rotate about the rotation axis can also be referred to herein as a pivot joint. The distal movement arm can be rotatable by at least 45°, preferably by at least 180° and particularly preferably by at least 360° about the rotation axis.

According to the invention, the rotation axis runs at an angle to the pivot axis. In other words, the rotation axis and the pivot axis are at an angle to each other that is greater than 0° and less than 180°. The angle between the rotation axis and the pivot axis can be in particular 70°, preferably 80° and particularly preferably 90°. The angle may deviate from a right angle within the scope of assembly and/or manufacturing tolerances and/or with a deviation of a maximum of 5°.

According to a development of the invention, the robotic apparatus can comprise an interactor and a coupling unit, wherein the coupling unit has a first coupling element connected to the interactor, a second coupling element connected to the distal movement arm, and a traction means arrangement with at least one traction means which is coupled to the first coupling element and the second coupling element, such that a movement can be transferred from one of the coupling elements to the other of the coupling elements. The interactor and/or the first coupling element can preferably be arranged close to the base, i.e. on and/or at the base of the robotic apparatus. Such a design allows a mass of the robotic apparatus to be concentrated closer to the base and thus significantly reduces the inertia of the robotic apparatus. The traction means arrangement makes it possible to efficiently transmit and/or absorb forces and/or movements between components that are further apart from one another, without structurally complex and cost-intensive gearing arrangements needing to be provided. Such a design can also help to improve ergonomics and reduce the risk of fatigue and/or resulting user errors.

An interactor can be understood in particular to mean a unit that is designed to absorb, transmit, measure and/or generate forces and/or movements. Additionally or alternatively, the interactor may provide a physical and/or virtual stop. The interactor can be operated and/or is operable in an active and/or passive mode. Depending on the application, the interactor can be operated and/or is operable exclusively in one of the two modes. For example, a robotic apparatus of a master unit can always be operated in a passive mode. A robotic apparatus of a slave unit can always be operated in an active mode.

A “passive mode” is understood to mean an operating state in which the robotic apparatus is passively driven, i.e. not actively driven; the robotic apparatus is moved by external forces, in particular by an attending physician and/or surgeon. For this mode, the interactor and/or the robotic apparatus may comprise a sensor system configured to capture movement data of the device while it is being moved manually, i.e. by hand. The movements and/or control signals can, for example, be interpreted by the sensor system of the robotic apparatus and converted into instructions that control motors and/or effectors. The control of the motors and/or effectors can preferably be carried out in real time with the input of movements, i.e. the movement of the robotic apparatus by a user. In the passive mode, one or more interactors can limit movements of joint sections and/or movement arms by means of one or more variable physical and/or virtual stops. The interactors may be configured to provide tactile feedback and/or resistance for a user.

The sensor system may comprise various measuring devices and/or sensors that appear advantageous to the person skilled in the art and that are designed to capture and interpret movements and/or changes in movement. The sensors can preferably be provided on the different joint sections and/or the rotary, pivot and/or rotation axes. The sensor system can in particular be encoders, gyroscope sensors, inertial sensors and/or Hall sensors, which are designed to detect and/or capture positions, movements, speeds and/or angular changes of the movement arms and/or coupling elements, preferably in real time. In this way, the input device enables the user to intuitively and/or precisely control the robotic apparatus and/or the effectors.

An “active mode” is understood herein to mean an operating state in which the robotic apparatus is actively driven and/or moved in order to perform a specific function. For this mode, the interactor and/or the robotic apparatus may comprise at least one drive source, for example in the form of an electric motor and associated gearing, in order to actively drive and/or move the robotic apparatus and/or sections thereof. In the active operating mode, the interactor and/or robotic apparatus can, in particular, navigate automatically and/or autonomously. Even in the active operating mode, the sensor system can be used to provide feedback about the environment of the robotic apparatus, monitor its movements and/or ensure that it performs its tasks properly.

Depending on the needs and/or requirements of the given task and/or user input, the robotic apparatus can switch and/or be switched between the active operating mode and the passive operating mode. In teleoperation systems, the master unit can preferably be operated in a passive operating mode. The slave unit can preferably be operated in an active operating mode.

A coupling unit can be understood herein as an apparatus and/or a system that is designed to couple different components of the robotic apparatus to one another mechanically and/or in a manner allowing the transmission of motion, which makes it possible for the coupled components to cooperate and/or interact with one another reliably and functionally during operation.

A coupling element can be defined in particular as a component that is designed to be coupled to a further coupling element in order to enable their interaction and/or function. In this case, the coupling element may comprise a type of drum which is designed to control and/or absorb movements of the traction means by the coupling element winding on and/or unwinding the traction means. The coupling element may preferably have a round cross-section and/or be cylindrical, such that the traction means can be at least partially wound on and/or unwound evenly and/or securely on different regions of the drum. The traction means may, for example, comprise a cord, a belt and/or a chain. According to some embodiments, the traction means may comprise a plurality of cords, belts and/or chains.

While the first coupling element can be arranged close to the base, the second coupling element can be arranged away from the base and/or the first coupling element. A distance can be provided between the first coupling element and the second coupling element which corresponds at least to the length of the proximal movement arm.

A traction means arrangement is intended to mean a structure and/or a mechanism that is designed to transmit forces and/or movements of the distal movement arm to the interactor and/or to the first coupling element and/or vice versa. The traction means arrangement can comprise multiple traction means. The traction means can interact with the coupling elements in such a way that winding on at least one traction means leads to the same traction means on the second coupling element unwinding, and vice versa.

In some embodiments, a first end portion of the traction means may be wound at least partially on and/or around the first coupling element and a second end portion of the traction means may be wound at least partially on and/or around the second coupling element, such that the first coupling element and the second coupling element are interdependently rotatably coupled. In this way, an efficient and/or synchronized coordination and/or cooperation of the coupling elements can be achieved.

“Interdependently rotatably coupled” is intended to mean in particular that the first coupling element can be coupled to the second coupling element in such a way that the two coupling elements influence each other, in particular with regard to their position and/or orientation. The position and/or behavior of each coupling element depends on the position and/or behavior of every other coupling element coupled in the coupling unit. The movements of the two coupling elements can be in the same direction. In other words, the first coupling element and the second coupling element interdependently coupled to the first coupling element can move synchronously. The coupling elements can be the same or different. The coupling elements can create a transmission ratio. The coupling elements may differ in particular in their size and/or diameter.

In order to hold the traction means in the correct position and to guide its movement, to ensure efficient power transmission and to utilize the installation space efficiently and/or in a space-saving manner, the traction means arrangement can comprise a plurality of guide means, preferably in the form of guide rollers. The guide means may comprise at least one groove and/or channel to guide the traction means and/or to redirect movements in a desired direction. Additionally or alternatively, the guide means may comprise pulleys and/or guide channels, for example in the form of guide cylinders. The arrangement of the guide rollers can guide the traction means along and/or on different reference planes that are at an angle to each other. In other words, the guide means can be arranged such that the traction means is or are guided on or parallel to a first reference plane, a second reference plane and/or at least one third reference plane, wherein the reference planes can extend obliquely and/or perpendicularly to one another. By using multiple guide means, the forces required to move the robotic apparatus can be reduced. This can, among other things, increase the user-friendliness and/or efficiency of the robotic apparatus.

The guide means can be arranged in such a way that the traction means crosses over itself at least partially. The traction means may comprise individual components, including, for example, cords, belts, chains and/or similar components and/or a combination thereof, each of which crosses over itself and/or with another component of the traction means. By crossing over, uneven load distributions along the guide rail can be compensated for, and the traction means can be guided in a space-saving manner.

The first coupling element and the second coupling element can each be rotatable about rotation axes which are arranged at an angle to one another, in particular at an angle greater than 0° and less than 180°, preferably greater than 80° and less than 100° and particularly preferably 90°. Rotation axes at an angle to each other allow a compact design of the robotic apparatus with multiple degrees of freedom and/or an efficient use of the installation space. Arrangement at different angles allows the robotic apparatus to execute complex application-specific movements precisely. In some embodiments, the rotation axes may be coaxial with the pivot axis and/or the rotation axis.

According to a further development of the invention, the distal movement arm can be pivotable relative to the proximal movement arm about a further pivot axis. The further pivot axis can run parallel to the pivot axis. The further pivot axis can preferably run at a 90° angle, i.e. orthogonal to the rotation axis. The angle may deviate from a right angle within the scope of assembly and/or manufacturing tolerances and/or with a deviation of a maximum of 5°.

The additional pivot axis allows the number of degrees of freedom of the robotic apparatus to be increased, which means that the robotic apparatus can be moved in more directions and with greater flexibility. In other words, the working range of the robotic apparatus can be increased. In this way, more complex and precise movements can be performed with high accuracy, which can lead to better treatment results, especially in surgery. The connection point that allows the proximal movement arm to pivot about the further pivot axis can also be referred to herein as an additional articulated joint.

In some embodiments, the robotic apparatus may comprise a side arm which extends at least partially, preferably largely, along and/or parallel to the proximal movement arm and is spaced therefrom. The side arm may be one or more movement elements designed to couple the base to the proximal movement arm and/or the distal movement arm. The side arm can be movable relative to the proximal movement arm when the distal movement arm pivots about the further pivot axis. Among other things, the side arm can improve the stability of the robotic apparatus and enable more precise movement control. Furthermore, loads can be distributed to the proximal movement arm and the side arm, which can have a positive effect on the service life and/or reliability of the robot mechanism. Furthermore, the side arm can help to increase the stability of the robotic apparatus and to minimize unwanted movements.

According to some embodiments, the traction means arrangement can be arranged at least partially on and/or in the side arm. In this way, a traction means arrangement can be ensured which is efficient and/or less susceptible to failure and/or is protected. Such an arrangement can facilitate maintenance and/or troubleshooting of the traction means arrangement.

According to a development of the invention, the robotic apparatus may comprise an input device which is configured to capture a movement and/or commands of a user. The input device may be arranged at a distal end portion of the distal movement arm. The input device may preferably be a device and/or an interface configured to transmit data and/or commands to the robotic apparatus. The input device may in particular comprise a joystick and/or control lever configured to precisely control the robotic apparatus in different directions and positions and/or to transmit control signals to a computer system and/or other devices. The input device can preferably be part of a master unit and/or can be functionally coupled to such a unit.

In alternative embodiments, instead of the input device an effector may be provided at the distal end of the distal movement arm. The effector may in particular comprise a medical instrument. The medical instrument may be any effector that appears appropriate to a person skilled in the art, in particular, in the form of a medical and/or surgical instrument, which can preferably be carried by the robotic apparatus and be movable thereby. The medical instrument may be permanently or releasably connected to the distal movement arm or may be part of the distal movement arm. The medical instrument may, for example, have a laparoscopic unit, an endoscope, a microscope, an exoscope, a scalpel, a drill, a scraper, a clamp, a pair of forceps, a pair of scissors, a syringe, a catheter and/or other units that appear appropriate to a person skilled in the art, which can be driven and/or actuated by means of the robotic apparatus. The effector can preferably be part of a slave unit and/or can be functionally coupled to such a unit.

In some embodiments, the robotic apparatus may comprise an orientation mechanism configured to prevent and/or compensate for a corresponding rotational movement of the input device and/or of the effector at least during a rotational movement of the distal movement arm about the rotation axis. This makes it easier and/or more reliable to achieve a desired and/or precise positioning of the input device and/or of the effector, which reduces errors and/or simplifies handling.

The orientation mechanism may comprise at least two pulleys, wherein a first pulley is arranged at a proximal end portion of the distal movement arm, and wherein a second pulley is arranged at the distal end portion of the distal movement arm. Furthermore, the orientation mechanism may comprise at least one traction element that is tensioned between and/or around the at least two pulleys in order to synchronize an orientation of the pulleys.

A pulley can be a disk which has at least one groove and/or one channel on its radial circumferential surface in order to guide the traction element.

According to some embodiments, the input device and/or the effector can be arranged in a rotationally fixed manner on the second pulley. This allows the distal movement arm to move without entraining the input device and/or the effector. This particularly improves the freedom of movement, which overall contributes to an efficient and/or user-friendly performance of the robotic apparatus.

In a development of the invention, the first pulley can be stationary relative to the rotation axis and the second pulley can be movable relative to the rotation axis. Such a configuration makes possible a rotationally fixed arrangement of the input device and/or the effector on the second pulley. This allows the input device and/or the effector to be moved independently of the rest of the robotic apparatus and/or vice versa. In other words, the orientation of the input device and/or of the effector may remain unaffected by a movement of the distal movement arm and/or of the robotic apparatus about one of the pivot axes.

The at least one traction element can be fixed to the pulleys at least one point. This fixation ensures that the orientation of the input device and/or of the effector remains unchanged after and/or during a movement of the distal movement arm and/or of the robotic apparatus about a rotation axis. The user and/or a servomotor do not need to apply any additional force to maintain the orientation of the input device and/or effector during use.

In addition or alternatively to the orientation mechanism, the robotic apparatus may comprise a further orientation mechanism. The further orientation mechanism may in particular comprise a joint device which is configured to align the input device and/or the effector independently of the orientation mechanism or of a movement of the distal movement arm about an orientation axis. For this purpose, the further orientation mechanism can comprise a push and/or pull rod. A push and/or pull rod may in particular be one or more rods which are designed to exert a compressive force and/or a tensile force on the joint device. Such a configuration has the advantage that a desired orientation and/or alignment of the input device and/or of the effector can be maintained even after and/or during a movement of the distal movement arm and/or of the robotic apparatus.

The robotic apparatus and/or individual components thereof may comprise a cladding. The cladding may, in particular, be designed to protect components that are at least partially surrounded by the cladding against external influences such as dust, moisture and/or mechanical damage. The cladding may be made of various materials such as plastic, metal, or rubber. The cladding may be configured so that it can be easily connected to at least one other housing or system in a captive manner.

In addition or alternatively to the interactor, the robotic apparatus may comprise at least one physical and/or virtual stop element configured to permit movements of the individual components about corresponding pivot and/or rotation axes of the respective components and/or to limit them after a certain travel path or magnitude. A virtual stop element can be understood, for example, to mean a programming of the robotic apparatus. In this way, movement ranges can be defined and/or modified easily and without the need to install additional hardware. In this way, unwanted loads or excessive movements and thus potential damage to the robotic apparatus can be prevented. The at least one stop element can be considered as a kind of protective mechanism to ensure the integrity and reliability of the robotic apparatus.

According to a further development, the robotic apparatus may comprise the base. The base can be rotatable about a further rotation axis. The further rotation axis creates an additional degree of freedom, which further increases the working field of the robotic apparatus.

The invention further relates to a medical robotic system comprising at least one robotic apparatus of the type described above.

Such a design makes it possible to provide an advantageously further developed robotic system. In particular, such a robotic system has low inertia while maintaining a high degree of freedom of movement. In addition, a robotic system can be provided with an advantageous ratio of the size of the robotic system to its working space, which can in particular increase the precision and/or the user-friendliness of the robotic system.

The robotic system may have at least one electronics cabinet, in particular an electronics rack, e.g. for accommodating additional devices such as an RF generator, a display unit, a patient couch, a storage unit for various medical instruments and/or further units that appear useful to the person skilled in the art. The medical robotic system may comprise multiple robotic apparatuses, in particular one for each arm of an operator. In an operating state, a medical instrument and/or a control console can each be carried by a robotic apparatus and can be moved by it.

The robotic system can comprise a display unit that is configured to display for a user an image, in particular a moving image of an object area to be processed. The object area may in particular be an area that includes physiological components, such as tissue, blood or the like. The object area is located, for example, within a natural or artificially created cavity. Such cavities include the abdominal cavity, the intestines, the bladder, the kidney, or the like. However, open tissue could also serve as an object area. The display unit can comprise a screen and/or control electronics. The display unit can comprise a computer and/or processor and/or memory and/or random access memory and/or ports and/or a data interface for receiving, processing and outputting unprocessed, preprocessed and/or processed image data and/or image data.

The devices and systems according to the invention and the methods according to the invention are hereby not to be limited to the application and embodiment described above. In particular, they can have a number of individual elements, components, and units which differ from a number mentioned herein, in order to fulfill a function described herein. In addition, for the ranges of values specified in this disclosure, values within the stated limits shall also be deemed to be disclosed and to be usable in any manner.

The present invention is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included but not a second object. However, a number and/or sequence of objects could also be derived using numerical words.

### BRIEF DESCRIPTION OF DRAWINGS

FIG. 1 is a medical robotic system with a robotic apparatus for controlling a medical instrument,

FIG. 2 is a perspective view of a detailed view of the robotic apparatus,

FIG. 3 is a perspective view of the robotic apparatus without cladding,

FIG. 4 is a perspective view of the robotic apparatus without cladding from a different angle,

FIG. 5 is a perspective view of the robotic apparatus with a traction means arrangement,

FIG. 6 is a schematic illustration of the traction means arrangement,

FIG. 7 is a perspective view of the distal movement arm in a first position,

FIG. 8 is a perspective view of the distal movement arm in a second position,

FIG. 9 is a schematic illustration of the distal movement arm in the first position,

FIG. 10 is a schematic illustration of the distal movement arm in the second position,

FIG. 11 is a side view of a robotic apparatus according to a further embodiment in a first position, and

FIG. 12 is a side view of the robotic apparatus according to FIG. 11 in a second position.

### DETAILED DESCRIPTION OF PREFERRED EMBODIMENTS

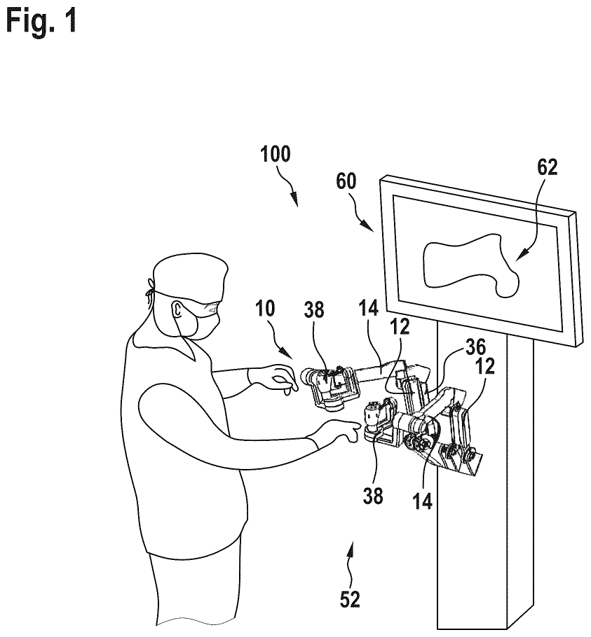

FIG. 1 shows a medical robotic system 100. The robotic system 100 comprises a master unit 52 and two robotic apparatuses 10, wherein the master unit 52 is configured to control a slave unit (not shown here) via a user. The robotic apparatuses 10 of the slave unit are each equipped with an input device 38 arranged on a distal movement arm, which is configured to receive a movement and/or a command from the user. At this point, it should be noted that the input devices 38 shown here are only shown schematically. It goes without saying that different input devices 38 can be used depending on the area of application. The medical robotic system 100 further comprises a display unit 60 which is configured to display to the user an image 62 of an object area to be processed.

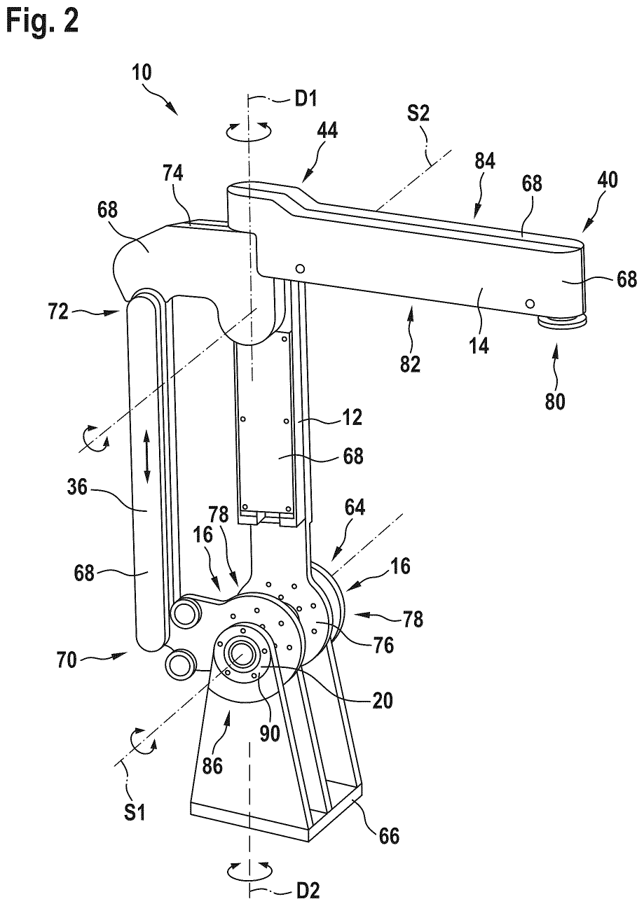

The robotic apparatuses 10 have multiple degrees of freedom, thereby covering a particularly large or broad working area. With regard to the structural design of the robotic apparatuses 10, reference can be made to FIGS. 2 to 12 below.

FIG. 2 shows a perspective view of a detailed view of one of the robotic apparatuses 10 according to FIG. 1. Since the robotic apparatuses 10 shown in FIG. 1 are constructed mirror-symmetrically to one another, the following statements apply to both robotic apparatuses 10 shown in FIG. 1. The robotic apparatus 10 comprises, in addition to the distal movement arm 14, a proximal movement arm 12. The proximal movement arm 12 has a proximal arm portion 64 which is coupled to a base 66 such that the proximal movement arm 12 can be pivoted about a pivot axis S1 and/or an articulated joint relative to the base 66.

The distal movement arm 14 further has a proximal end portion 44 which is coupled to the proximal movement arm 12 via an articulation unit 74, such that the distal movement arm 14 can be pivoted about a rotation axis D1 and/or a pivot joint relative to the proximal movement arm 12. The rotation axis D1 runs perpendicular to the pivot axis S1.

The robotic apparatus 10 has a side arm 36 with a proximal arm portion 70 and a distal arm portion 72. The proximal arm portion 70 of the side arm 36 is articulated to the base 66 via a further articulation unit 76. The distal arm portion 72 of the side arm 36 is coupled to the articulation unit 74. The side arm 36 and the proximal movement arm 12 run essentially parallel to each other. The side arm 36 and the proximal movement arm 12 are spaced apart from each other.

The side arm 36 can be moved up and down independently of the proximal movement arm 12. This degree of freedom of the side arm 36 is indicated by an arrow in FIG. 2. Such an upward or downward movement of the side arm 36 can pivot the distal movement arm 14 about a further pivot axis S2 and/or a further articulated joint relative to the proximal movement arm 14. The further pivot axis S2 runs parallel to the pivot axis S1. The base 66 of the robotic apparatus 10 can be rotatable about a further rotation axis D2. The further rotation axis D2 can run parallel to the rotation axis D1. The further rotation axis D2 and the rotation axis D1 can run on a common axis.

The robotic apparatus 10 comprises multiple interactors 16 with sensor systems 78. The sensor system 78 is in particular designed to detect and/or capture movements and/or changes in movement of the robotic apparatus 10. The interactors 16 can be designed in such a way as to provide a physical and/or virtual stop in order to permit movements of the individual components of the robotic apparatus 10 about a corresponding pivot and/or rotation axis S1, S2, D1, D2 of the respective components and/or to limit them after a certain travel path or magnitude.

Furthermore, the robotic apparatus 10 comprises an interface 80 which is configured to be coupled to the input device 38. The input device 38 is not shown in FIG. 2. According to FIG. 2, the interface 80 is arranged on an underside 82 of the distal movement arm 14 in an end portion 40 of the distal movement arm 14. In other embodiments not shown here, the interface 80 may also be arranged at a different location on the distal movement arm 14, such as a top side 84 of the distal movement arm 14.

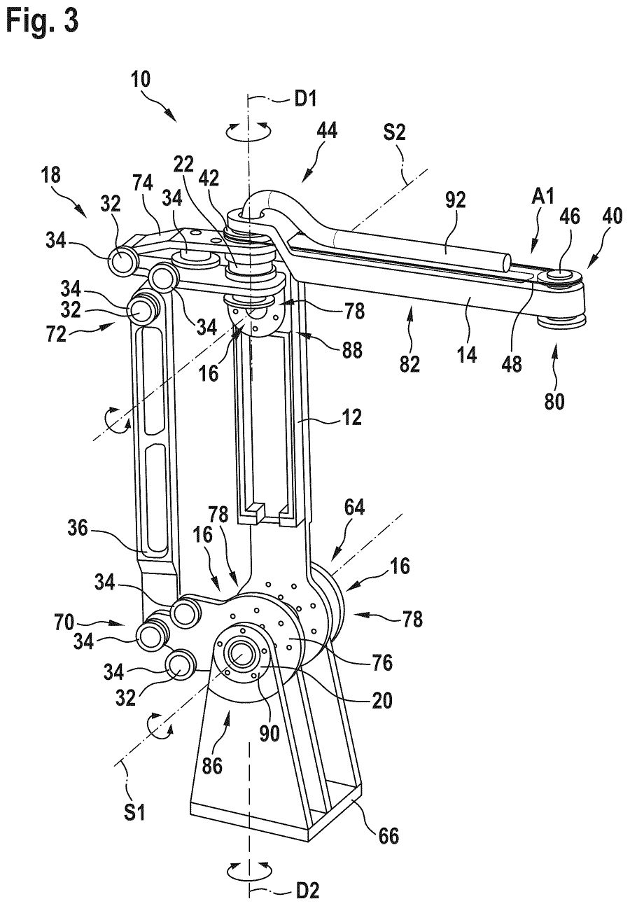

Due to cladding elements 68, the internal mechanical components of the robotic apparatus 10 cannot be seen in FIG. 2 except for a first coupling element 20. In this regard, reference is made to the following description of FIGS. 3 to 12.

FIG. 3 is a perspective view of the robotic apparatus 10 without cladding 68. A coupling unit 18 can be seen which has a second coupling element 22 in addition to the first coupling element 20. While the first coupling element 20 is arranged on a distal portion 86 of the base 66, the second coupling element 22 is arranged on a distal arm portion 88 of the proximal movement arm 12, or in other words between the distal arm portion 88 of the proximal movement arm 12 and the proximal end portion 44 of the distal movement arm 14. The second coupling element 22 is arranged on the articulation unit 74.

Furthermore, the robotic apparatus 10 comprises a traction means arrangement 24 with a plurality of guide means 32, in the form of guide rollers 34, which are configured to guide a traction means 26. In the embodiment shown here, the traction means arrangement 24 has a total of seven guide rollers 34. The guide rollers 34 are arranged such that the traction means 26 can be guided on different reference planes, wherein the reference planes are perpendicular to each other. The first coupling element 20 and the second coupling element 22 are configured as cylindrical cable drums 90 and are designed to be coupled to the traction means 26 (not shown in this figure) so that a movement can be transferred from one of the coupling elements 20, 22 to the other coupling element 20, 22.

Also visible is a cable conduit 92 which extends at least predominantly along the distal movement arm 14. The cable conduit 92 can in particular be configured to guide cables and/or lines, in particular mechanical, electrical and/or optical lines, securely to the interface 80 and/or to protect them from external influences.

The robotic apparatus 10 further comprises an orientation mechanism A1 which is configured to prevent and/or compensate for a corresponding rotational movement of a device which can be arranged on the distal movement arm 14, such as an input device 38, at least during a rotational movement of the distal movement arm 14 about the rotation axis D1.

For this purpose, the orientation mechanism A1 comprises at least two pulleys 42, 46, wherein a first pulley 42 is arranged at a proximal end portion 44 of the distal movement arm 14, and wherein a second pulley 46 is arranged at the distal end portion 40 of the distal movement arm 14.

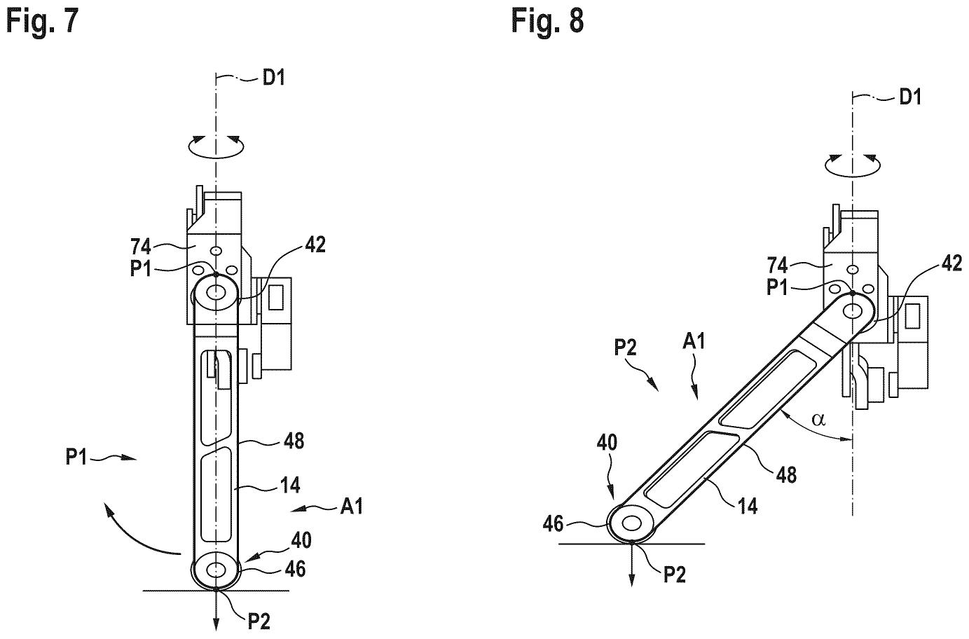

The orientation mechanism A1 also comprises at least one traction element 48 in the form of a cord which is stretched between the two pulleys 42, 46 in order to synchronize an orientation of the pulleys 42, 46. The first pulley 42 is stationary relative to the rotation axis D1. The second pulley 46 is movable relative to the rotation axis D1. The at least one traction element 48 is fixed to the pulleys 42, 46 at least one point P1, P2. With regard to the operating principle of the orientation mechanism A1, reference can also be made to FIGS. 7 to 10.

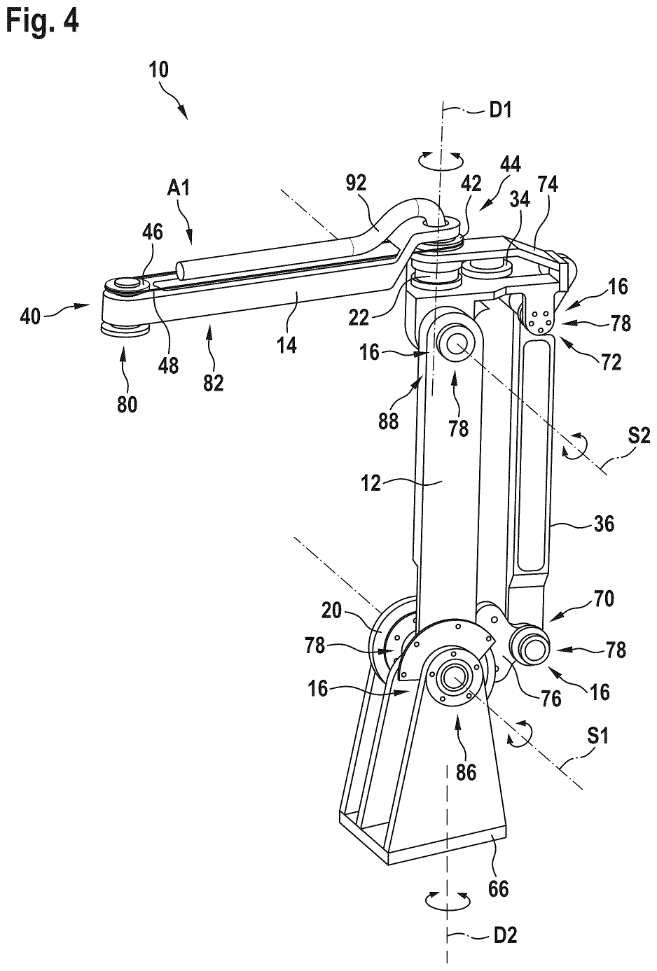

FIG. 4 is a perspective view of the robotic apparatus 10 without cladding from a different angle.

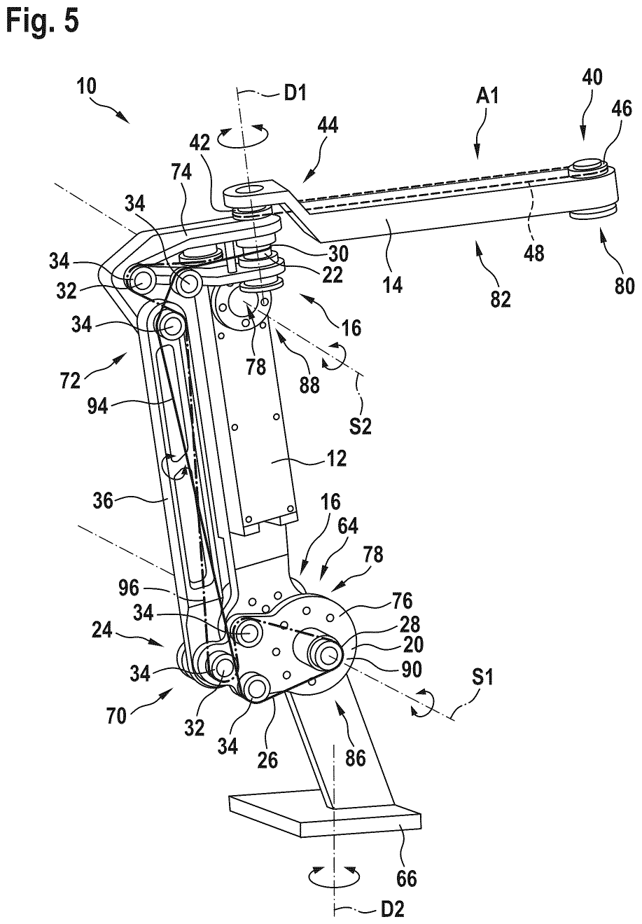

FIG. 5 is a perspective view of the robotic apparatus 10 with a traction means 26 consisting of a first and a second cord 94, 96. A first end portion 28 of each cord 94, 96 is at least partially wound around the first coupling element 20 and a second end portion 30 of each cord 94, 96 is at least partially wound around the second coupling element 22, such that the first coupling element 20 and the second coupling element 22 are interdependently rotatably coupled.

The traction means arrangement 24 is arranged at least partially on and/or in the side arm 36 and the articulation units 74, 76.

For efficient and space-saving force transmission between the coupling elements 20, 22, the traction means 26 is guided around the guide rollers 34. Each of the articulation units 74 and the further articulation unit 76 comprises three guide rollers, and each forms an articulated joint. Each guide roller 34 changes the direction of a cord 94, 96 deflected on the corresponding guide roller 34.

The guide rollers 34 are arranged such that the traction means 26 is guided crosswise at least partially. The two cords 94, 96 are guided in a counter-rotating manner in order to achieve overall a constant length of the traction means 26 and a constant winding angle of the traction means 26 around the first and second coupling elements 20, 22. If the winding angle of the traction means 26 is reduced due to a movement of the robotic apparatus 10 around one of the coupling elements 20, 22, the winding angle of the traction means 26 around the other coupling element 20, 22 increases.

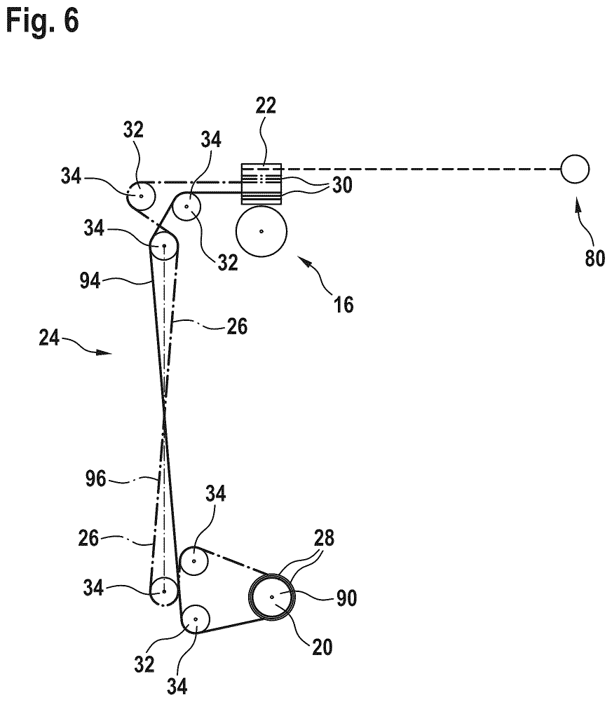

For better understanding, a schematic illustration of the traction means arrangement 24 is shown in FIG. 6.

FIG. 7 is a perspective view of the distal movement arm 14 in a first position P1. FIG. 8 is a perspective view of the distal movement arm 14 in a second position P2. The second position P2 differs from the first position P1 in that starting from the first position P1 the distal movement arm 14 has been pivoted about the rotation axis D1. It can be seen that by means of the orientation mechanism A1, a constant or identical orientation of the interface 80 and thus of an effector 58 and/or an input device 38 potentially arranged thereon can always be ensured during operation and/or movement of the robotic apparatus. As already described above with reference to FIG. 5, the first pulley 42 is stationary relative to the rotation axis D1. The second pulley 46 is movable relative to the rotation axis D1. The at least one traction element 48 is fixed to the pulleys 42, 46 at least one point P1, P2. The total winding angle of the traction element 48 on each of the first and second pulleys 42, 46 is 180°. As soon as the distal movement arm 14 is pivoted about the rotation axis D1, the part in which the traction element 48 is wound around one of the pulleys 42, 46 and/or contacts the same moves in a direction opposite to the pivoting direction.

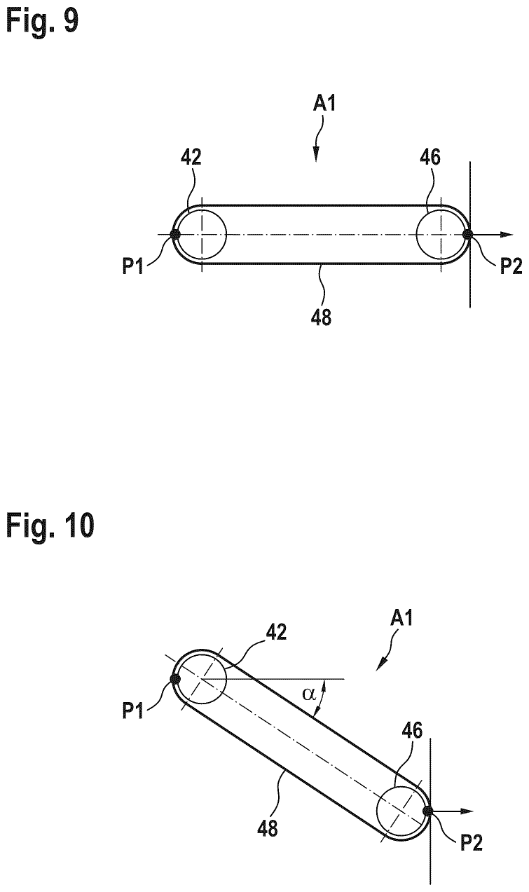

For better illustration, the functional principle of the orientation mechanism A1 is shown schematically again in FIGS. 9 and 10. FIG. 9 shows a schematic illustration of the orientation mechanism A1 in the first position P1. FIG. 10 shows a schematic illustration of the orientation mechanism in the second position P2, in which the distal movement arm 14 has been pivoted by an angular value a.

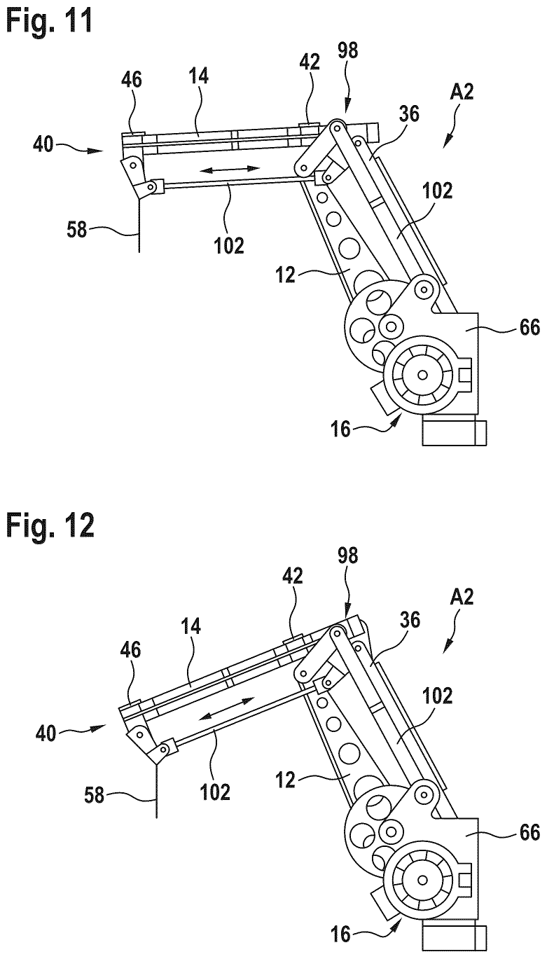

FIGS. 11 and 12 each show a side view of a robotic apparatus 10 according to a further embodiment. The embodiment shown in FIGS. 11 and 12 is in particular designed for use in a slave unit. According to this embodiment, the robotic apparatus 10 comprises an orientation mechanism A2 as an alternative to the orientation mechanism A1 described above. This orientation mechanism A2 uses special parallel kinematics to align the orientation of an effector coupled to the robotic apparatus 10 and/or to keep the orientation of the effector 10 constant with respect to a user, regardless of the positioning of the movement arms 12, 14 of the robotic apparatus 10. For this purpose, the orientation mechanism A2 comprises a pull and/or push rod arrangement 98. The pull and/or push rod arrangement 98 may comprise a plurality of pull and/or push rods 102 which are coupled to one another and to the effector 58 in a manner which transmits movement. The pull and/or push rod arrangement 98 may extend from a base 66 of the robotic apparatus 10 and along and/or parallel to a proximal and/or distal movement arm 14.

The operating principle of the further orientation mechanism A2 is shown in FIGS. 11 and 12. By means of linear movements of the pull and/or push rods 102, the effector 58 can be oriented independently of the movement arms 12, 14.

1. Robotic apparatus comprising:
a proximal movement arm which is pivotable about at least one pivot axis;
a distal movement arm which is articulately coupled to the proximal movement arm ,
wherein the distal movement arm is rotatable about at least one rotation axis relative to the proximal movement arm, wherein the rotation axis extends at an angle to the pivot axis.

2. Robotic apparatus according to claim 1, further comprising
an interactor and
a coupling unit which has a first coupling element connected to the interactor, a second coupling element connected to the distal movement arm, and a traction means arrangement with at least one traction means which is coupled to the first coupling element and the second coupling element, such that a movement can be transferred from one of the coupling elements to the other of the coupling elements.

3. Robotic apparatus according to claim 2, wherein a first end portion of the traction means is wound at least partially around the first coupling element and a second end portion of the traction means is wound at least partially around the second coupling element, such that the first coupling element and the second coupling element are interdependently rotatably coupled.

4. Robotic apparatus according to claim 2, wherein the traction means arrangement comprises a plurality of guide means, preferably guide rollers, which are adapted to guide the traction means.

5. Robotic apparatus according to claim 4, wherein the guide means are arranged such that the traction means crosses over itself at least in sections.

6. Robotic apparatus according to claim 2, wherein the first coupling element and the second coupling element are each rotatable about rotation axes which are arranged at an angle to one another.

7. Robotic apparatus according to any of the preceding claim 1, wherein the distal movement arm is pivotable relative to the proximal movement arm about a further pivot axis.

8. Robotic apparatus according to claim 7, wherein the further pivot axis runs parallel to the pivot axis.

9. Robotic apparatus according to claim 7, wherein the further pivot axis runs perpendicular to the rotation axis.

10. Robotic apparatus according to claim 7, further: including;
a side arm which runs at least partially along the proximal movement arm and is spaced therefrom and which is movable relative to the proximal movement arm during a pivoting movement of the distal movement arm about the further pivot axis.

11. Robotic apparatus according to claim 2, wherein the traction means arrangement is arranged at least partially on and/or in the side arm.

12. Robotic apparatus according to any of the preceding claim 1, further including:
an input device configured to capture a movement and/or commands from a user, wherein the input device is arranged on a distal end portion of the distal movement arm.

13. Robotic apparatus according to claim 12, further including:
an orientation mechanism which is configured to prevent and/or compensate for a corresponding rotational movement of the input device at least during a rotational movement of the distal movement arm about the rotation axis.

14. Robotic apparatus according to claim 13, wherein the orientation mechanism includes:
at least two pulleys, wherein a first pulley is arranged at a proximal end portion of the distal movement arm, and wherein a second pulley is arranged at the distal end portion of the distal movement arm;
at least one traction element which is tensioned between and/or around the at least two pulleys in order to synchronize an orientation of the pulleys.

15. Robotic apparatus according to claim 12, wherein the input device is arranged rotationally fixed on the second pulley.

16. Robotic apparatus according to claim 14, wherein the first pulley is stationary relative to the rotation axis and the second pulley is movable relative to the rotation axis.

17. Robotic apparatus according to claim 14, wherein the at least one traction element is fixed to the pulleys at at least one point.

18. Medical robotic system comprising:
a robotic apparatus according to claim 1.
